(19) United States
(12) Patent Application Publication
(10) Pub. No.: US 2011/0306799 A1
(43) Pub. Date: Dec. 15, 2011

(54) METHOD FOR THE TARGETED EXECUTION OF A REACTION TAKING PLACE ALONGSIDE SEVERAL COMPETING CHEMICAL REACTIONS

(76) Inventors: Rainer Schenk, Kelsterbach (DE); Michael Küpper, Mainz (DE); Thomas Schwalbe, Bad Vilbel (DE)

(21) Appl. No.: 12/307,489

(22) PCT Filed: May 25, 2007
(86) PCT No.: PCT/EP2007/004673

§ 371 Date: Jun. 10, 2011

(30) Foreign Application Priority Data
Jul. 8, 2006 (DE) 10 2006 031 652.5

(51) Int. Cl. C07C 45/29

(52) U.S. Cl. 568/376

(57) ABSTRACT

A method for the targeted execution of a reaction taking place alongside several competing chemical reactions is described, wherein the speeds of the competing reactions are set by the selection of the reaction conditions in such a way that the desired main reaction is not adversely affected. The invention further describes a new method for the oxidation of primary or secondary alcohols into aldehydes or ketones in the presence of dimethyl sulfoxide, a carboxylic acid anhydride or halogenide, and an amine in an organic solvent, in a continuous processing system (Moffatt-Swern oxidation).

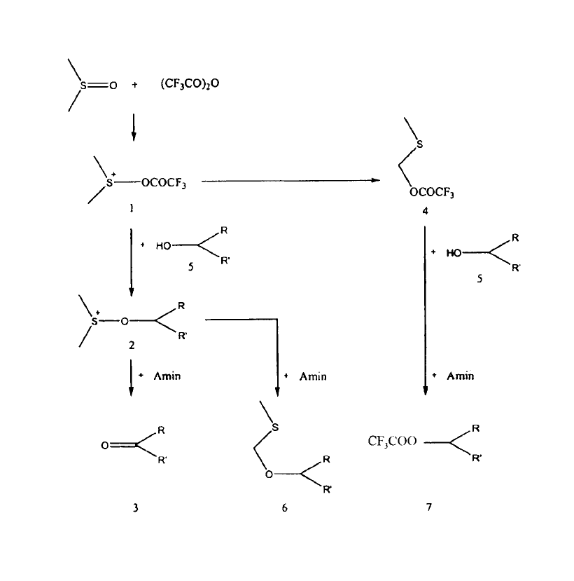

The invention provides a process for controlled performance of a reaction which proceeds alongside a plurality of competing chemical reactions. In addition is a novel process for oxidizing primary or secondary alcohols to aldehydes or ketones in the presence of dimethyl sulfoxide, a carboxylic anhydride or carbonyl halide and an amine in an organic solvent in a continuous process system.

The Moffatt-Swern oxidation of alcohols to carbonyl compounds is one of the most versatile and reliable methods for this conversion (K. Omura, A. K. Sharma, D. Swern, J. Org. Chem. 1976, 41, 957-962; K. Omura, D. Swern, Tetrahedron 1978, 34, 1651-1660; A. K. Sharma, D: Swern, Tetrahedron Lett. 1974, 1503-1506; A. K. Sharma, T. Ku, A. D. Dawson, D. Swern, J. Org. Chem. 1975, 40, 2758-2764). It is frequently utilized for organic synthesis on the laboratory scale (T. T. Tidwell, Org. React. 1990, 39, 297-572). In this oxidation, the conversion of the alcohol/dimethyl sulfoxide redox pair to carbonyl compound/dimethyl sulfide is associated with an activation by organic acid anhydrides or halides. The latter firstly accelerate the reaction and secondly afford the exergonic driving force owing to the formation of an ammonium salt from the organic acids released. The proposed mechanism of the Moffat-Swern oxidation includes a sequence of three successive reactions, the second and the third being accompanied by parallel and competing reactions (figure). These sometimes parallel and competing reactions lead to the formation of by-products which can use up the alcoholic starting material.

The batchwise synthesis protocol employed to date begins with the addition of an organic acid anhydride, for example trifluoroacetic anhydride (TFAA) in methylene chloride (CH₂Cl₂) to a solution of dimethyl sulfoxide (DMSO) in CH₂Cl₂ at or below −50° C. After this addition has ended, the alcoholic starting material is added dropwise at the same temperature. Subsequently, a base, usually triethylamine, is then likewise added dropwise at the same temperature.

In order to conclude the conversion of the intermediate 2 to the final product, the mixture is warmed to room temperature.

The above-described protocol, characterized by the sequential addition at low temperature, is firstly employed because the undesired side reaction of the intermediate 1 to give the intermediate by-product 4 proceeds sufficiently slowly at low temperatures in order to ensure enrichment of the desired intermediate 1 during the overall procedure of addition of TFAA or in the course of subsequent addition of the alcoholic starting material. It has been stated that the conversion of the intermediate 1 to the intermediate by-product 4, known as the Pummerer rearrangement, proceeds very rapidly at temperatures above −30° C. when the batchwise synthesis is employed (K. Omura, A. K. Sharma, D. Swern, J. Org. Chem. 1976, 41, 957-962).

Secondly, the intermediate 2 can be rearranged to the undesired by-product 6. It has been proposed that prolonged reaction times at low temperatures and a retarded reaction of the intermediate 2 to give the desired end product 3 enhance the conversion to the undesired by-product 6 (T. Kawaguchi, H. Miyata, K. Ataka, K. Mae, J. Yoshida, Angew. Chem. Int. Ed. 2005, 44, 2413-2416). One conceivable explanation for this would be, irrespective of the more favorable rate constant, a higher activation enthalpy for the desired reaction of 2 to give 3 compared to the conversion of 2 to 6.

Thirdly, it can be assumed that, after the addition of the alcoholic starting material to the solution of 1 and before the addition of the base, the intermediate 1 has already been converted fully to the desired intermediate 2, as demonstrated by the number of applications of the batch protocol in organic synthesis on the laboratory scale. No intermediate 1 should therefore be present any longer for the Pummerer rearrangement to 4, which is base-induced and catalyzed, in contrast to the thermal rearrangement.

The provision of the cooling for the industrial scale limits the selection of the reaction apparatus, requires expensive infrastructure for the supply with coolants and additionally gives rise to high energy costs. Moreover, in the adjustment of this protocol to larger production units, the addition time of TFAA to DMSO has to be extended, in order to maintain the target temperature among other reasons, as a result of which more time is available for the undesired side reaction of 1 to give 4. All process sizes in the addition of the base and the subsequent reaction of 2 to give 3 which are designed to promote the desired reaction path compared to the competing route to 6 are again dependent on the type and size of the components used in the batch apparatus.

One adjustment of the Swern-Moffatt oxidation to a continuous process regime has already been described (T. Kawaguchi, H. Miyata, K. Ataka, K. Mae, J. Yoshida, Angew. Chem. Int. Ed. 2005, 44, 2413-2416), using microreactors (Microreaction Technology (Ed.: W. Ehrfeld), Springer, Berlin, 1998; W. Ehrfeld, V. Hessel, H. Loewe, Microreactors, Wiley-VCH, Weinheim, 2000; Microsystem Technology in Chemistry and Life Sciences (Eds.: A. Manz, H. Becker), Springer, Berlin, 1999; V. Hessel, S. Hardt, H. Loewe, Chemical micro Process Engineering, Wiley-VCH, Weinheim, 2004). The utilization of microreactors in organic synthesis has already been described many times (K. Jaehnisch, V. Hessel, H. Loewe, M. Baerns, Angew. Chem. Int. Ed. 2004, 116, 4, 410-451; P. Fletcher, S. Haswell, E. Pombo-Villar, B. Warrington, P. Watts, S. Wong, X. Zhang, Tetrahedron, 2002, 58, 4735-4756; H. Pennemann, P. Watts, S. Haswell, V. Hessel, H. Loewe, Org. Process Res. Dev. 2004, 8, 3, 422-439; S. Taghavi-Moghadam, A. Kleemann, K. Golbig, Org. Process Res. Dev. 2002, 5, 652-659; T. Schwalbe, V. Autze, G. Wille, Chimia, 2002, 56, 636-646). The effect of the microreactor arrangement on the process control is the subject of many publications with the aim of promoting a new synthesis method (T. Schwalbe, V. Autze, M. Hohmann, W. Stirner, Org. Process Res. Dev. 2004, 8, 440-454; V. Hessel, P. Loeb, H. Loewe, Cur. Org. Chem. 2005, 9, 765-787; T. Schwalbe, K. Simons, Chimica Oggi—Chemistry Today, 2006, 24, 56-61).

The adjustment, described by Kawaguchi et al., 2005, of the Swern-Moffatt oxidation to a continuous process regime follows the batch protocol very strictly in the addition of the reagents. In this case, a solution of DMSO (4.0M, 2 eq.) in CH₂Cl₂ is mixed with a solution of TFAA (2.4M, 1.2 eq.) in CH₂Cl₂, and the reaction is performed at a temperature of from −20 to 20° C. within 0.01-2.4 s. This solution is then mixed with a solution of the alcoholic starting compound (1M) in CH₂Cl₂ and the reaction is carried out at the same temperature for 1.2 s. At the same temperature again, this solution was then mixed with a solution of triethylamine (1.45M, 2.9 eq.) in CH₂Cl₂ and the reaction was carried out for 1.2 s. Finally, the mixture was warmed to 30° C. for 5.9 s. Various alcohols were oxidized in this way with a high yield and selectivity to carbonyl compounds.

This protocol profits from the strict control of the temperature, as a result of which an undesired accelerated degradation of 11s reduced, combined with the strict control of the lifetime of 1 owing to the rapid transfer of the reactive solution to the second mixing operation with the alcoholic starting compound. The subsequent steps are also performed in such a way that they profit from these two control factors.

The combination of these two developed measures for control enables the process regime at room temperature. Since this procedure is stable over prolonged operating times, it is possible in principle to convert greater amounts by this process.

However, this process nevertheless has some disadvantages. The overall process arrangement requires at least four pumps and three or more reactors. Such a process regime leads to considerable expenditure on the industrial scale, caused by the necessity of providing appropriate infrastructure and maintenance and repair measures. The reaction times of the individual steps are defined by the particular volumes based on the flow rates. This experimental arrangement is therefore suitable very specifically for one preparation process which requires the conditions implemented therein. Because not only the reaction volumes but also the reaction times are determined by the flow rates, this experimental arrangement is suitable only for a very limited range of reaction times for a given throughput or, vice versa, for a defined throughput.

The object consists in the provision of a process which enables a chemical reaction sequence to be carried out with sufficient yield to give the desired end product in spite of a plurality of competing reactions. The aim is especially to reduce the number of reactors required for a continuous process and to maximize their permissible flow, and to find a shortened addition protocol compared to the batch process, in order to minimize the number of and requirement for cooling steps and residence times, and the formation of temperature-dependent unstable intermediates.

The object is achieved by adjusting the rates of the competing reactions through the selection of the reaction conditions such that the desired main reaction is not impaired. The process according to the invention described here is based on the creation of a rapid mixing sequence with a smallest possible combination of one or more mixing points for a maximum possible kinetic concurrence of the individual reagents and the conversion products thereof in the particular reaction zone in terms of space and time.

It is additionally advantageous to suppress undesired competing reactions by rapid mixing of the reactants.

In this process, undesired competing reactions can preferably be suppressed by close temperature control.

It is additionally advantageous that a rapid scavenging reaction removes the intermediate which leads to the end product from the reaction mixture.

It is additionally advantageous that, by virtue of a final scavenging reaction, the end product is present in a solution in which it does not enter into any subsequent reactions under the present conditions.

It is additionally particularly advantageous when the reactions are optionally carried out partially or completely in a micromixer or microreactor. The individual reactions may optionally also be carried out in conventional reaction vessels.

For the first time, conditions under which particular mixtures of different reagents with the starting substance of the Moffatt-Swern oxidation react with one another in no other way than along the path which leads to the aim of the oxidation have now been found. For instance, it has specifically been found that, from a mixture of DMSO and an alcohol, the DMSO reacts preferentially with TFAA and other organic anhydrides and acid halides. It has additionally been found that DMSO does not react with alcohols and that a mixture of DMSO, alcohols and amines does not react with itself. This is one of the boundary conditions which is crucial for the configuration of the reaction regime, which has been found by means of ¹H NMR studies. More particularly, there were no indications that DMSO and an alcohol, in the presence or absence of an amine, form an equilibrium in solution with a thio hemiketal. It has nevertheless been demonstrated that a solution of DMSO, alcohol and amine, when mixed with a solution of TFAA and other organic anhydrides, leads to some degree to the oxidation product according to the Swern-Moffatt oxidation. In addition, TFAA and other organic anhydrides react in the expected exothermic reaction with triethylamine and other tertiary amines to give trifluoroacylammonium triflates or equivalent ammonium salts. However, it has been found that these salts react similarly to the acid anhydrides. Consequently, individual starting substances of the Moffatt-Swern oxidation can be mixed actually before the start of the reaction, which allows significantly simplified process regimes to be realized.

This gives rise to the advantageous possibility of a process for oxidation of primary or secondary alcohols to aldehydes or ketones in the presence of dimethyl sulfoxide, a carboxylic anhydride or carbonyl halide, and an amine in an organic solvent in a continuous process system, where this reaction sequence up to the commencement of the final oxidation step can be carried out under the action of the amine at a temperature between −30° C. and +50° C. within a period of less than 10 s, and at least two of the substances are mixed before the start of the reaction and the aldehyde formed or the ketone is isolated after the temperature is adjusted to room temperature. In this type of process regime, temperatures between −20° C. and +20° C. have been found to be particularly advantageous.

It has been found to be particularly suitable to use trifluoroacetic anhydride (TFAA) as the carboxylic anhydride. A preferred organic solvent component used for the primary or secondary alcohols, the dimethyl sulfoxide, the carboxylic anhydride or carbonyl halide and the amine is methylene chloride.

In this process, preference may be given to adding a solution consisting of the primary or secondary alcohol and dimethyl sulfoxide in solvent to a solution of TFAA, and then to adding the amine in solvent. Particularly advantageously, the process can be carried out partially or completely in a micromixer or microreactor. Methylene chloride is a preferred organic solvent. To this end, in a first step, a solution of DMSO and alcohol in organic solution is first mixed in a continuous microreactor process with a solution of TFAA in CH₂Cl₂. Subsequently, the reaction is effected by adding a solution of a tertiary amine in organic solution. This forms aldehydes with a high excess relative to the by-product 7. In this configuration of the process as a continuous two-step process, at least one reaction vessel can be dispensed with compared to the conventional process regime (see example 1).

In addition, it is advantageous to add a solution consisting of the primary or secondary alcohol and dimethyl sulfoxide in solvent, optionally in a micromixer or microreactor, to a solution of TFAA, and then to carry out the addition of the amine or solution thereof in a conventional reaction vessel. Methylene chloride is a preferred organic solvent. Such a configuration constitutes a continuous one-step process with subsequent batch aminolysis. The aldehydes are likewise obtained in a high excess relative to the by-product 7. It is particularly advantageous here that the dissolved reaction mixture composed of originally alcohol, dimethyl sulfoxide and TFAA can be transferred via a non-temperature-controlled line to a conventional reaction vessel for the purpose of addition of the amine solution. The reaction can then be carried out at room temperature therein. This gives rise to considerable savings potential with regard to the process systems to be provided (see example 2).

A further advantageous variant of the process consists in a combination of the solution consisting of the primary or secondary alcohol and dimethyl sulfoxide in solvent and a mixture of the amine and TFAA. The process can particularly advantageously be performed partially or completely in a microreactor. Methylene chloride is a preferred organic solvent. For this purpose, a solution of DMSO and alcohol in solvent is mixed in a continuous microreactor process with a solution of TFAA and amine. This forms predominantly aldehydes or ketones as well as readily measurable amounts of by-product 7. In this continuous one-step process, the advantage arises, which should be emphasized, of a possible reduction of the plants to only one reaction unit (see example 3).

The process further enables a type of process regime in which a solution consisting of the primary or secondary alcohol, dimethyl sulfoxide and the amine in solvent is added to a solution of TFAA. The process can particularly advantageously be performed partially or completely in a micromixer or microreactor. Methylene chloride is a preferred organic solvent. By means of a continuous one-step process, a solution of DMSO, alcohol and the amine in solvent is mixed in a continuous microreactor process with a solution of TFAA in an organic solvent, for example CH₂Cl₂ (see example 4).

In addition, the possibility arises of configuring the process such that a solution consisting of the primary or secondary alcohol, dimethyl sulfoxide and an amine in solvent is added to a solution of TFAA and an amine in an organic solvent, for example CH₂Cl₂. The process can particularly advantageously be carried out partially or completely in a micromixer or microreactor. Methylene chloride is a preferred organic solvent.

In addition, the possibility arises of replacing dimethyl sulfoxide with other dialkyl sulfoxides, the reaction products of which are less volatile, and which therefore leads only to crude product solutions with reduced odor nuisance. In addition, cyanuric chloride can be used instead of dimethyl sulfoxide.

The Moffatt-Swern oxidation of alcohols to carbonyl compounds is one of the most versatile and reliable methods for this conversion. This oxidation is used very widely in industry, for example in the preparation of pharmaceutical intermediates, but to date always at very low temperatures in order to avoid the undesired Pummerer rearrangements. The employment of the process according to the invention allows a reduction in the number of reactors, cooling steps and residence times required, and a minimization of the temperature-dependent formation of unstable intermediates. A process regime which is thus considerably simplified leads, through dispensing with the requirement for pumps, reactors and cooling apparatus, to a considerable potential for cost savings.

### EXPLANATIONS FOR THE FIGURE

FIGURE Reaction routes of the Moffatt-Swern oxidation. In this FIGURE, 1 to 7 characterize the following compounds: 1 trifluoroacetoxy-dimethylsulfonium ion, 2 alkoxydimethylsulfonium ion, 3 carbonyl compound, 4 thiomethylmethyl trifluoroacetate, 5 alcohol, 6 alkoxymethyl methyl sulfide, 7 trifluoroacetic ester

### EXAMPLES

### Example 1

### Two-Step Continuous Process

Solution 1: TFAA (0.6M) in CH₂Cl₂; flow rate: 10 ml/min
Solution 2: cyclohexanol (0.5M) and DMSO (1M) in CH₂Cl₂; flow rate: 10 ml/min
Solution 3: tributylamine (0.73M) in CH₂Cl₂; flow rate: 20 ml/min

The continuous process was carried out in a 2-step microreaction system. In step 1, solutions 1 and 2 were mixed in a micromixer with a residence time of 2 s. The reaction was effected both at a temperature of −20° C. and of +20° C. Subsequently, this mixture was mixed with solution 3 in a micromixer (step 2), for which the residence time was 4 s and the temperature was identical to step 1. Thereafter, the entire solution was warmed to room temperature and stored for a few minutes before the mixture was analyzed by gas chromatography (GC). A yield of 91% of cyclohexanone was obtained.

### Example 2

### One-Step Continuous Process with Batch Aminolysis

Solution 1: TFAA (0.6M) in CH₂Cl₂; flow rate: 15 ml/min
Solution 2: cyclohexanol (0.5M) and DMSO (1M) in CH₂Cl₂; flow rate: 15 ml/min
Solution 3: tributylamine (0.73M) in CH₂Cl₂; flow rate: 30 ml/min

In a continuous process, solutions 1 and 2 were mixed with a micromixer with a residence time of 1.5 s (step 1). The reaction was effected both at a temperature of −20° C. and of +20° C. Subsequently, the reaction mixture was transferred through a non-temperature-controlled line (with a residence time of 5 s) into a conventional reaction vessel (step 2). At the same time, solution 3 was also pumped into this vessel and mixed conventionally at room temperature for 1.5 min. A total reaction volume of 90 ml was collected. The yield of cyclohexanone (93%) in this example was comparable to the yield of example 1.

### Example 3

### One-Step Continuous Process

Solution 1: TFAA (0.3M) and tributylamine (0.73M) in CH₂Cl₂; flow rate: 40 ml/min
Solution 2: cyclohexanol (0.5M) and DMSO (1M) in CH₂Cl₂; flow rate: 20 ml/min

In a continuous process, solutions 1 and 2 were mixed using a micromixer in a 1-step microreaction system at a temperature of −30° C. Thereafter, the entire reaction solution was warmed to room temperature and stored for a few minutes before the mixture was analyzed by GC. In this process, the yield of cyclohexanone was below 45%.

### Example 4

### One-Step Continuous Process

Solution 1: TFAA (0.6M) in CH₂Cl₂; flow rate: 20 ml/min
Solution 2: cyclohexanol (0.5M), DMSO (1M) and tributylamine (1.43M) in CH₂Cl₂; flow rate: 20 ml/min

In a continuous process, solutions 1 and 2 were mixed using a micromixer in a 1-step microreaction system. The reaction was carried out both at a temperature of −20° C. and of +20° C. Thereafter, the entire reaction solution was warmed to room temperature and stored for a few minutes before the mixture was analyzed by GC. The content of cyclohexanone was substantially independent of the temperature. In this process, the yield of cyclohexanone was 20%.

1. A process for controlled performance of a reaction which proceeds alongside a plurality of competing chemical reactions, comprising selecting reaction conditions to adjust the rate of competing reactions so that the desired main reaction is not impaired.

2. The process as claimed in claim 1, wherein said conditions comprise rapid mixing of the reactants to suppress undesired competing reactions.

3. The process as claimed in claim 1, wherein said conditions comprise close temperature control to suppress undesired competing reactions.

4. The process as claimed in claim 1, wherein said conditions comprise a rapid scavenging reaction to remove the intermediate which leads to the end product from the reaction mixture.

5. The process as claimed in claim 1, wherein said conditions comprise a final scavenging reaction whereby the end product is present in a solution in which it does not enter into any subsequent reactions under the present conditions.

6. The process as claimed in claim 1 wherein, the reactions are optionally carried out partially or completely in a micromixer or microreactor.

7. The process as claimed in claim 1 for oxidation of primary or secondary alcohols to aldehydes or ketones in the presence of dimethyl sulfoxide, a carboxylic anhydride or carbonyl halide and an amine in an organic solvent in a continuous process system, wherein the underlying reaction sequence up to the commencement of the final oxidation step can be carried out under the action of the amine at a temperature between −30° C. and +50° C. within a period of less than 10 s, and at least two of the substances are mixed before the start of the reaction and the aldehyde formed or the ketone is isolated after the temperature is adjusted to room temperature.

8. The process as claimed in claim 7, wherein the carboxylic anhydride used is trifluoroacetic anhydride (TFAA).

9. The process as claimed in claim 7, wherein methylene chloride is used as the organic solvent.

10. The process as claimed in claim 7, wherein a solution consisting of the primary or secondary alcohol and dimethyl sulfoxide is added to a solution of TFAA and then the amine is added.

11. The process as claimed in claim 7, wherein a solution consisting of the primary or secondary alcohol and dimethyl sulfoxide, optionally in a micromixer or microreactor, is added to a solution of TFAA and then, in a conventional reaction vessel, the amine or solution thereof is added.

12. The process as claimed in claim 7, wherein a solution consisting of the primary or secondary alcohol and dimethyl sulfoxide is added to a solution of the amine and TFAA.

13. The process as claimed in claim 7, wherein a solution consisting of the primary or secondary alcohol, dimethyl sulfoxide and the amine is added to a solution of TFAA.

14. The process as claimed in claim 7, wherein a solution consisting of the primary or secondary alcohol, dimethyl sulfoxide and the amine is added to a solution of TFAA and the amine.

15. The process as claimed in claim 7, wherein another dialkyl sulfoxide is used instead of dimethyl sufoxide.

16. The process as claimed in claim 7, wherein cyanuric chloride is used instead of dimethyl sulfoxide.
